(19) United States
(12) Patent Application Publication
(10) Pub. No.: US 2005/0197661 A1
(43) Pub. Date: Sep. 8, 2005

(54) Tissue removal probe with sliding burr in cutting window

(75) Inventors: Harold F. Carrison, Pleasanton, CA (US); Scott McGill, San Ramon, CA (US); Leonardo R. Novero, San Jose, CA (US); Gopal K. Chopra, Seattle, WA (US); Ajitkumar B. Nair, Milpitas, CA (US)

Correspondence Address: Bingham McCuthen, LLP, Suite 1800, Three Embarcadero, San Francisco, CA, 94111-4067, US

(73) Assignee: Scimed Life Systems, Inc., Maple Grove, MN (US)

(21) Appl. No.: 10/793,693

(22) Filed: Mar. 3, 2004

(51) Int. Cl. A61B 17/32

(52) U.S. Cl. 606/79; 606/170

(57) ABSTRACT

A probe and method for removing tissue is provided. The probe comprises an elongated member, a window laterally formed on the distal end of the member, a drive shaft rotatably disposed within the lumen of the member, and a rotatable and longitudinally slidable tissue removal element disposed on the drive shaft. The probe can be used to remove tissue along the window of the probe. In one method, target tissue along the window, e.g., bone tissue, can be removed without removing non-target tissue, e.g., nerve tissue, by rotating and longitudinally sliding the tissue removal element relative to the window.

### RELATED APPLICATIONS

This application is related to copending applications Ser. No. 10/xxx,xxx (Attorney Docket Nos. 2024730-7038292001,), Ser. No. 10/xxx,xxx (Attorney Docket No. 2024730-7036832001) and Ser. No. 10/xxx,xxx (Attorney Docket No. 2024730-7038282001), which are expressly incorporated herein by reference.

### FIELD OF THE INVENTION

The field of the invention pertains to medical devices and methods for removing tissue, and in particular, bone tissue, such as vertebral bone tissue.

### BACKGROUND OF THE INVENTION

The spinal column consists of thirty-three bones called vertebra, the first twenty-four vertebrae of which make up the cervical, thoracic, and lumbar regions of the spine and are separated from each other by “pads” of tough cartilage called “intervertebral discs,” which act as shock absorbers that provide flexibility, stability, and pain-free movement of the spine.

FIGS. 1 and 2 illustrate a portion of a healthy and normal spine, and specifically, two vertebra 10 and two intervertebral discs 12 (only one shown). The posterior of the vertebra 10 includes right and left transverse processes 14R, 14L, right and left superior articular processes 16R, 16L, and a spinous process 18. Muscles and ligaments that move and stabilize the vertebra 10 are connected to these structures. The vertebra 10 further includes a centrally located lamina 20 with right and left lamina 20R, 20L, that lie inbetween the spinous process 18 and the superior articular processes 16R, 16L. Right and left pedicles 22R, 22L are positioned anterior to the right and left transverse processes 14R, 14L, respectively. The lamina 20 (vertebral arch 24) extends between the pedicles 22. The anterior of the vertebra 10 includes a vertebral body 26, which joins the vertebral arch 24 at the pedicles 22. The vertebral body 26 includes an interior volume of reticulated, cancellous bone (not shown) enclosed by a compact cortical bone 30 around the exterior. The vertebral arch 24 and vertebral body 26 make up the spinal canal (i.e., the vertebral foramen 32), which is the opening through which the spinal cord 34 and epidural veins (not shown) pass. Nerve roots 36 laterally pass from the spinal cord 34 out through the neural foramen 38 at the side of the spinal canal formed between the pedicles 22. Structurally, the intervertebral disc 12 consists of two parts: an inner gel-like nucleus (nucleus pulposus) 40 located centrally within the disc 12, and tough fibrous outer annulus (annulus fibrosis) 42 surrounding the nucleus 40.

A person may develop any one of a variety of debilitating spinal conditions and diseases. For example, as illustrated in FIG. 3, when the outer wall of the disc 12′ (i.e., the annulus fibrosis 42) becomes weakened through age or injury, it may tear allowing the soft inner part of the disc 12 (i.e., the nucleus pulposus 40) to bulge out, forming a herniation 46. The herniated disc 12′ often pinches or compresses the adjacent nerve root 36 against a portion of the vertebra 10, resulting in weakness, tingling, numbness, or pain in the back, leg or arm areas.

Often, inflammation from disc herniation can be treated successfully by nonsurgical means, such as bedrest, therapeutic exercise, oral anti-inflammatory medications or epidural injection of corticosterioids, and anesthetics. In some cases, however, the disc tissue is irreparably damaged, in which case, surgery is the best option.

Discectomy, which involves removing all, or a portion, of the affected disc, is the most common surgical treatment for ruptured or herniated discs of the lumbar spine. In most cases, a laminotomy or laminectomy is performed to visualize and access the affected disc. Once the vertebrae, disc, and other surrounding structures can be visualized, the surgeon will remove the section of the disc that is protruding from the disc wall and any other offending disc fragments that may have been expelled from the disc. In some cases, the entire disc may be removed, with or without a bony fusion or arthroplasty (disc nucleus replacement or total disc replacement).

Besides disc hernias, other debilitating spinal conditions or diseases may occur. For example, spinal stenosis, which results from hypertrophy proximate to a vertebra, reduces the space within the spinal canal, compromising or displacing canal contents. When the nerve roots 36 are pinched, a painful, burning, tingling, and/or numbing sensation is felt down the lower back, down legs, and sometimes in the feet. As illustrated in FIG. 2, the spinal canal 32 has a rounded triangular shape that holds the spinal cord 34 without pinching. The nerve roots 36 leave the spinal canal 32 through the neural foramen 38, which should be free of obstruction. As shown in FIG. 4, new bone growth 48 (e.g., bone spurs) within the spinal canal 32, and specifically from the diseased lamina 20 and facets, causes compression of the nerve roots, which may lead to painful spinal stenosis. Spinal stenosis may be treated by performing a decompression (laminectomy, foraminotomy, etc.) in order to relieve pressure on the nerve root 36 impinged by the bone growth 48. Depending on the extent of the bone growth, the entire lamina and spinal process may be removed.

Thus, it can be appreciated that in many spinal treatment procedures, bone and/or disc tissue must be removed in order to release pressure from neural tissue or rebuild the vertebra. In the case of target bone tissue that is adjacent spinal tissue, a physician is required to exercise extreme care when cutting away the target bone tissue (e.g., during a laminectomy and foraminotomy), such that injury to spinal tissue can be prevented. A physician may have difficulty controlling existing bone removal devices, however, and may unintentionally remove healthy bone tissue or injure spinal tissue during use. This problem is exacerbated with percutaneous treatments, which, although less invasive than other procedures, limit the range of motion of the cutting instrument, thereby further limiting the control that the physician may have during the bone cutting procedure.

Furthermore, during a bone cutting process, a media, such as saline, is generally delivered via a tube to a target site for clearing debris. The delivered media together with the debris are then removed from the target site via a separate tube (i.e., the media and the debris are aspired into a vacuum port of the tube). However, certain target sites, such as a vertebra, may not have enough room to accommodate both the bone removal device and the tubes.

There, thus, remains a need to provide for improved tissue removal probes and methods for use during spinal treatment and other surgeries.

### SUMMARY OF THE INVENTION

In accordance with a first aspect of the present inventions, a tissue removal probe is provided. The tissue removal probe comprises an elongated member (such as, a sleeve) having a lumen and a distal end. The member may be rigid or flexible, and the member distal end may be curved. If flexible, the member distal end may be steerable. The tissue removal probe further comprises a window laterally formed on the member distal end, a drive shaft rotatably disposed within the member lumen, and a rotatable tissue removal element (e.g., an abrasive burr) disposed on the drive shaft. The tissue removal element is longitudinally slidable within the window. Although the invention should not be so limited in its broadest aspects, the longitudinally slidable tissue removal element may be rotated and axially displaced to remove tissue without moving the member shaft. In one embodiment, the tissue removal probe can have a handle mounted to the member. In this case, the handle can be mated with a drive unit. In another embodiment, the tissue removal probe can comprise a tissue separator disposed on the distal end of the member shaft.

The tissue removal element can be supported within the window in any one of a variety of manners. In one embodiment, the tissue removal probe comprises a bearing affixed within the member lumen. In this case, the drive shaft has a rigid distal end, which can be formed as part of the tissue removal element, slidably disposed within the bearing. Or the tissue removal probe can comprise a housing slidably mounted within the window, e.g., in a sliding rail and groove arrangement. In this case, the tissue removal element can be rotatably disposed within the housing. Or, the tissue removal probe can have a guidewire extending along the window. In this case, the tissue removal element is configured to slide along the guidewire. For example, the tissue removal element can have a lumen through which the guidewire extends.

In accordance with a second aspect of the present inventions, a method of removing target tissue, e.g., bone tissue, without removing non-target tissue, e.g., nerve tissue, is provided. In one method, the target tissue is a vertebral lamina, and the non-target tissue is nerve tissue, e.g., tissue pertinent to a laminectomy procedure. The method comprises providing a probe with a window and a tissue removal element disposed in the window, and placing the window against the tissue. In one method, the target tissue is separated from the non-target tissue prior to placing the probe therebetween. In another method, the non-target tissue is shielded from the tissue removal element. The method further comprises rotating and longitudinally sliding the tissue removal element relative to the window to remove the target tissue along the window.

### BRIEF DESCRIPTION OF DRAWINGS

The drawings illustrate the design and utility of preferred embodiments of the present invention, in which similar elements are referred to by common reference numerals. In order to better appreciate how the above-recited and other advantages and objects of the present inventions are obtained, a more particular description of the present inventions briefly described above will be rendered by reference to specific embodiments thereof, which are illustrated in the accompanying drawings. Understanding that these drawings depict only typical embodiments of the invention and are not therefore to be considered limiting of its scope, the invention will be described and explained with additional specificity and detail through the use of the accompanying drawings in which:

FIG. 1 is a perspective view of a portion of a spine;

FIG. 2 is a top view of a vertebra with a healthy intervertebral disc;

FIG. 3 is a top view of a vertebra with a herniated intervertebral disc;

FIG. 4 is a top view of a vertebra with spinal stenosis;

FIG. 5 is a perspective view of a tissue removal probe constructed in accordance with a preferred embodiment of the present invention;

FIG. 6 is a partially cutaway side view of the distal end of the probe of FIG. 5, particularly showing the tissue removal element in its fully proximal position;

FIG. 7 is a partially cutaway side view of the distal end of the probe of FIG. 5, particularly showing the tissue removal element in its fully distal position;

FIG. 8 is a cross-sectional view of the tissue removal probe of FIG. 6, taken along the line 8-8;

FIG. 9 is a partially cutaway top view of the distal end of the probe of FIG. 5;

FIG. 10 is a side view of an alternative rotatable distal end of the probe of FIG. 5;

FIG. 11 is a side view of another alternative rotatable distal end of the probe of FIG. 5;

FIG. 12 is a partially cutaway side view of an alternative steerable distal end of the probe of FIG. 5;

FIG. 13 is a side view of an alternative tissue removal element that can be used in the tissue removal probe of FIG. 5;

FIG. 14 is a cross-sectional view of the tissue removal element of FIG. 13, taken along the line 13-13;

FIG. 15 is a partially cutaway side view of the distal end of the probe of FIG. 5, particularly showing an alternative means of supporting the tissue removal element in its proximal position;

FIG. 16 is a partially cutaway side view of the distal end of the probe of FIG. 15, particularly showing the tissue removal element in its distal position;

FIG. 17 is a cross-sectional view of the distal end of the probe of FIG. 15, taken along the line 17-17;

FIG. 18 is a partially cutaway side view of the distal end of the probe of FIG. 5, particularly showing another alternative means of supporting the tissue removal element in its proximal position;

FIG. 19 is a partially cutaway side view of the distal end of the probe of FIG. 18, particularly showing the tissue removal element in its distal position;

FIG. 20 is a side view of the distal end of the probe of FIG. 5, particularly showing an alternative tissue separator;

FIG. 21 is a top view of the distal end of the probe of FIG. 20;

FIG. 22 is a perspective view of a tissue removal probe constructed in accordance with another preferred embodiment of the present invention;

FIG. 23 is a partially cutaway top view of the distal end of the probe of FIG. 22;

FIG. 24 is a cross-sectional view of the tissue removal probe of FIG. 23, taken along the line 24-24;

FIG. 25A is a lateral view showing the introduction of the tissue removal probe of FIG. 5 through a passage adjacent the lamina of a vertebra;

FIG. 25B is a lateral view showing the use of the tissue removal probe of FIG. 5 in separating a nerve root from the lamina;

FIG. 25C is a superior view showing the placement of the distal end of the tissue removal probe between the lamina and the nerve root;

FIG. 25D is a superior view showing the use of the tissue removal probe of FIG. 5 in removing a linear inner surface portion of bone tissue from the lamina;

FIG. 25E is a superior view showing the use of the tissue removal probe of FIG. 5 in removing the entire linear thickness of bone tissue from the lamina;

FIG. 25F is a posterior view showing the use of the tissue removal probe of FIG. 5 in removing the entire linear thickness of bone tissue from the lamina; and

FIG. 25G is a posterior view showing the use of the tissue removal probe of FIG. 5 in removing another entire linear thickness of bone tissue from the lamina.

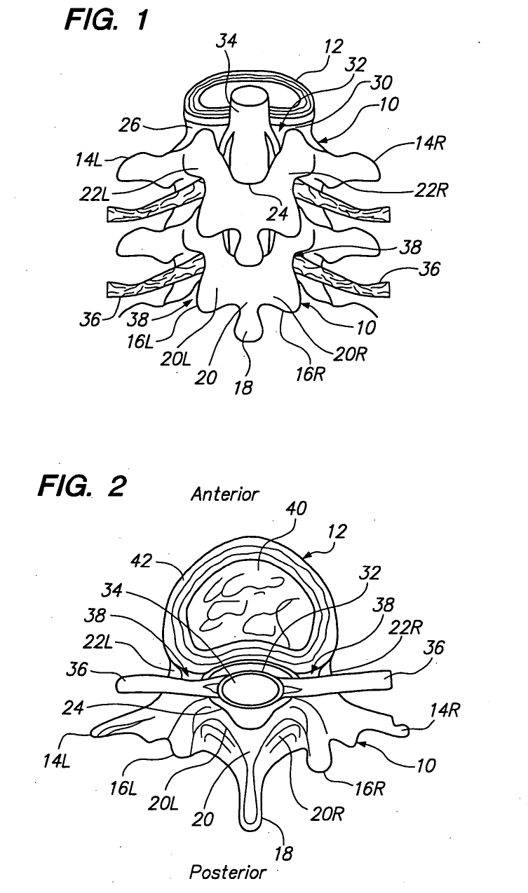

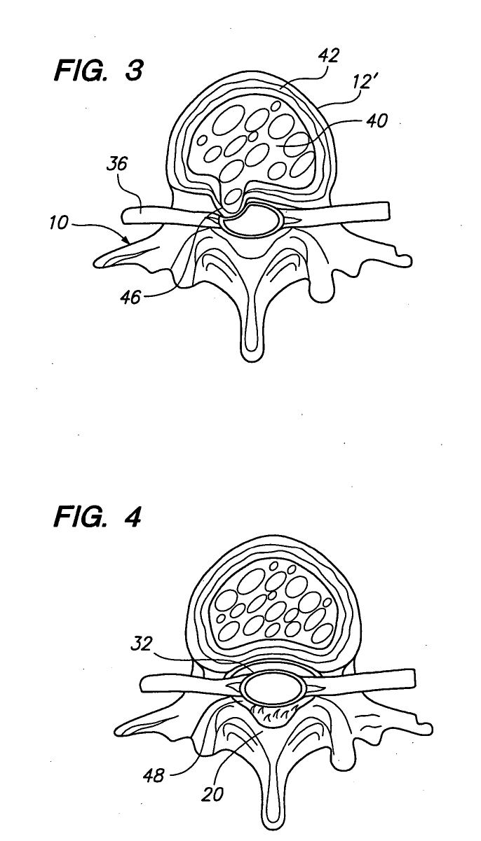

### DETAILED DESCRIPTION OF EMBODIMENTS

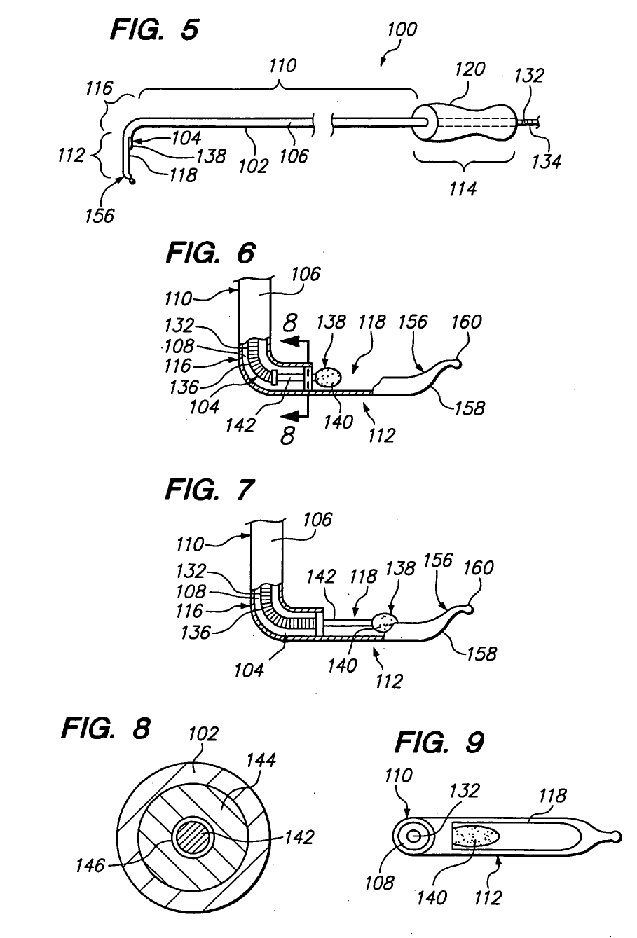

FIGS. 5-9 illustrate a tissue removal probe 100 constructed in accordance with a preferred embodiment of the present inventions. The probe 100 generally comprises an outer sleeve 102 and a tissue removal core 104 rotatably and slidably disposed within the outer sleeve 102.

The sleeve 102 comprises a hollow shaft 106 and a lumen 108 extending through the shaft 106 for receiving the tissue removal core 104. The shaft has a relatively long straight portion 110, a distal end 112 in which there is laterally formed a tissue-cutting window 118, and a proximal end 114 (shown in phantom in FIG. 5) on which there is mounted a handle 120. The distal end 112 of the sleeve shaft 106. The sleeve shaft 106 further has a curved portion 116 between the straight portion 110 and a distal end 112, which, as will be described in further detail below, allows tissue to be removed in a plane that is not parallel to the entry path through the tissue. In the illustrated embodiment, the curved portion 116 defines a 90 degree arc, which allows the tissue to be more efficiently removed in a plane that is perpendicular to the entry path. The curved portion 116 may define other arcs, depending on the angle formed between the tissue removal plane and the entry path. If the entry path lies in the tissue removal plane, the sleeve shaft 106 can be entirely straight, in which case, the curved portion may be eliminated.

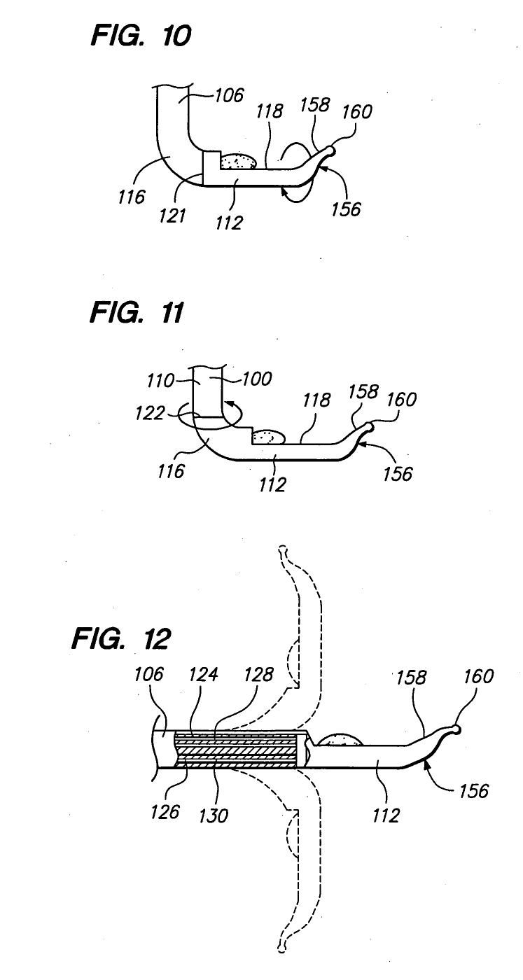

Alternatively, as shown in FIG. 10, the distal end 112 of the sleeve shaft 106 can be rotatably attached to the curved portion 116 of the sleeve shaft 106 at an interface 121 (e.g., in a snap-fit configuration), such that the cutting window 118 can be rotated about its axis (shown by arrow). Or, as shown in FIG. 11, the distal end 112 and curved portion 116, as a single piece, can be rotatably attached to the straight portion 110 of the sleeve shaft 106 at an interface 122, such that the cutting window 118 can be rotated about the axis of the straight portion 110 of the shaft 106 (shown by arrow).

In any event, the outer diameter of the outer sleeve shaft 106 is preferably less than ½ inch, but other dimensions for the outer diameter of the outer sleeve shaft 106 may also be appropriate, depending on the particular application or clinical procedure. The outer shaft lumen 108 should have an inner diameter so as to allow the tissue removal core 104 to be rotatably and slidably housed therein, as will be described in further detail below.

To facilitate placement and maintenance of the cutting window 118 at the tissue removal site, the outer sleeve shaft 106 is preferably rigid (e.g., it can be composed of a rigid material, or reinforced with a coating or a coil to control the amount of flexing), so that the outer sleeve 102 provides a more stable platform from which to remove tissue. Depending on the application, however, the entire sleeve shaft 106, or a portion thereof, can be composed of a flexible or malleable material, thereby allowing a physician to bend the tissue removal probe 100 into a desired shape during use. The materials used in constructing the outer sleeve shaft 106 may comprise any of a wide variety of biocompatible materials. In one embodiment, a radiopaque material, such as metal (e.g., stainless steel, titanium alloys, or cobalt alloys) or a polymer (e.g., ultra high molecular weight polyethylene) may be used, as is well known in the art.

In the case where the sleeve shaft 106 is composed of a flexible or malleable material, the tissue removal probe 100 may have optional steering capability. For example, in FIG. 12, the tissue removal probe 100, which has an entirely straight sleeve shaft 106, and thus no curved portion 116 when relaxed, comprises two steering wires 124, 126 that extends through a pair of steering wire lumens 128, 130 extending along the length of the sleeve shaft 106. The steering wires 124, 126 are distally secured to a bearing 144 (described in further detail below) mounted within the distal end of the shaft lumen 108, and proximally terminate in a steering mechanism (not shown) within the handle 120. In the case where the bearing 144 does not exist, the steering wires 124, 126 can be mounted to a ring (not shown) mounted within the wall of the shaft 106.

The steering wires 124, 126 extend down opposite sides of the sleeve shaft 106 and terminate at opposite sides of the bearing 144 (or otherwise a ring), such that when the steering wire 124 is pulled proximally, tension in the steering wire 124 causes the distal end 112 of the sleeve shaft 106 to bend in one direction (shown upward in phantom) from its normally straight configuration, and when the steering wire 126 is pulled proximally, tension in the steering wire 126 causes the distal end 112 of the sleeve shaft 106 to bend in the opposite direction from its normally straight configuration (shown downward in phantom).

It should be noted that the number of steering wires can be different from two. For example, in alternative embodiments, the outer sleeve 102 can have only one steering wire, thereby allowing the distal end 112 of the sleeve shaft 106 to be steered (or bent) in one direction only. In other embodiments, the outer sleeve 102 can have more than two steering wires coupled to the distal end 112 of the sleeve shaft 106 at different radial positions, thereby allowing the distal end 112 of the outer sleeve 102 to bend in multiple planes. In addition, it should be noted that the steering wire can be secured to the sleeve shaft 106 at different locations along its length. Furthermore, the manner in which the steering wire(s) is secured to the sleeve shaft 106 should not be limited to the foregoing example. In alternative embodiments, the steering wire(s) can be secured to a leaf spring (opposite sides of the leaf spring if two steering wires are used) longitudinally extending through the sleeve shaft 106.

Returning to FIGS. 5-9, the tissue removal core 104 comprises a drive shaft 132 having a proximal end 134 (shown only in FIG. 5) and a distal end 136, and a tissue removal element 138 mounted to the distal end 136 of the drive shaft 132. In the illustrated embodiment, drive shaft 132 is made of a flexible material, such as coiled or braided stainless steel. The tissue removal element 138 comprises an abrasive burr 140 and a rigid proximally extending shaft 142 that is suitably mounted to the distal end 136 of the drive shaft 132 by a connection using means such as a welding, brazing, or glue, depending on the material from which the burr shaft 142 and the drive shaft 132 are made. Alternatively, the burr shaft 142 can be secured to the drive shaft 132 by a snap-fit connection, a screw connection, or an interference-fit connection.

In the illustrated embodiment, the burr 140 includes abrasive particles, such as diamond dust, that are disposed on a surface of the burr 140. In other embodiments, instead of, or in addition to, having diamond dust, parts of the surface of the burr 140 can be removed to create an abrasive surface. The burr 140 can also include one or more grooves formed along the surface of the burr 140. In such case, the groove(s) allows bone particles that have been removed to travel proximally and away from a target site. The burr 140 is preferably made from a tough material, such as steel or other alloys, so that it could penetrate or cut into bone tissue without being damaged.

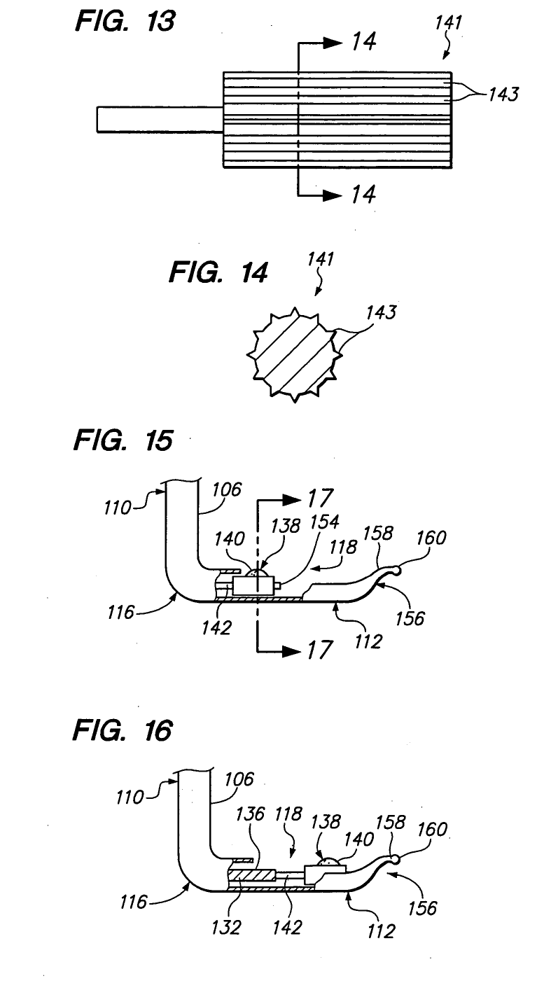

As shown in FIGS. 6, 7 and 9, the burr 140 has an elliptical profile. Alternatively, the burr 140 can have other shapes, such as a spherical shape or a cylindrical shape. For example, FIGS. 13 and 14 illustrate another burr 141 that can be used instead of the burr 140. The burr 141 has a cylindrical shape and a plurality of longitudinally cutting teeth 143 circumferentially disposed around the burr 141. Burrs that can be used with the probe 100 should not be limited to the foregoing examples, and may have a variety of shapes, sizes, and configurations, so long as the burr is capable of cutting, deforming, and/or abrading a target bone tissue.

In some embodiments, a cutting basket (not shown) can be used instead of the burr 140. In such cases, the cutting basket can be made from filaments having sharp edges, thereby providing bone cutting/drilling capability. In other embodiments, the cutting basket includes abrasive particles, such as diamond dust, disposed on surfaces of the filaments, for cutting, digging, and/or sanding against target bone tissue. In some embodiments, the cutting basket can be made from a resiliently elastic metal, such as nitinol.

As best shown in FIGS. 6, 7 and 9, the cutting window 118 exposes a portion of the burr 140, such that the burr 140 cuts and abrades bone tissue only on one lateral side (top) of the tissue removal probe 100, while protecting tissue at the opposite lateral side (bottom) of the tissue removal probe 100. As best shown in FIG. 9, the cutting window 118 has a rectangular shape, but can have other shapes as well. As can be appreciated, longitudinal movement of the drive shaft 132 within the outer shaft lumen 108, in turn, slides the burr 140 along the cutting window 118 between a proximal position (FIG. 6) and a distal position (FIG. 7). As such, the cutting window 118 advantageously limits the tissue removed to that which extends along the cutting window 118. At the same time, the length of the cutting window 118 allows a length of tissue to be removed without having to move the sleeve 102. The length of the cutting window 118 will depend upon the length of the tissue that is to be removed. In the illustrated embodiment, the length of the cutting window 118 is in the range of 0.25″-1.5″.

In order ensure that the burr 140 remains within the periphery of the cutting window 118, and can smoothly be slid therein, a cylindrical bearing 144 is suitably affixed within the outer shaft lumen 108 (shown in FIG. 8) just proximal to the cutting window 118. The bearing 144 comprises an aperture 146 through which the burr shaft 142 can slide. The size of the bearing aperture 146 is slightly larger than the diameter of the burr shaft 142, so that there is a snug fit between the burr shaft 142 and the bearing aperture 146. In this manner, the burr 140 can slide within the cutting window 118 without pitching.

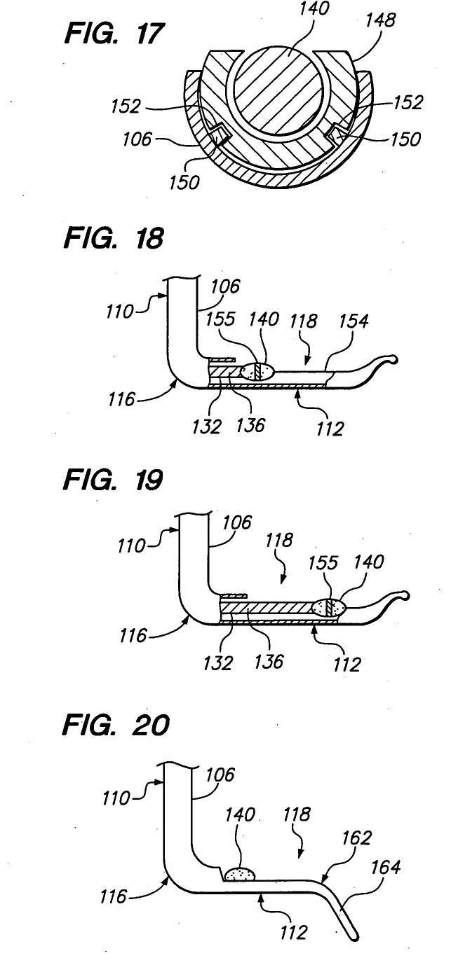

Alternatively, rather than using a bearing, the burr 140 can be rotatably disposed within a housing 148 that slides within the distal end 112 of the sleeve shaft 106 along the cutting window 118, as illustrated in FIGS. 15 and 16. In particular, as best shown in FIG. 17, the inner surface of the distal end 112 of the sleeve shaft 106 comprises a pair of rails 150, and the outer surface of the housing 148 comprises a pair of corresponding grooves 152 that slidably engage the respective rails 150. Alternatively, the inner surface of the distal end 112 of the sleeve shaft 106 may comprise rails, and the outer surface of the housing 148 may have grooves that slidably engage each other. The burr shaft 142 proximally extends through an opening (not shown) in the proximal end of the housing 148, and is mounted to the distal end 112 of the drive shaft 132 in the same manner described above. The distal end of the burr 140 comprises a peg 154 (shown in FIG. 15) that extends through an opening (not shown) in the distal end of the housing 148. Alternatively, the distal end of the burr 140 can have a hole, and the distal end of the housing 148 can have a peg that mates with the hole in the burr. In any event, the burr 140 is axially supported on both sides of the housing 148 to ensure that the burr 140 rotates about a stable axis. As can be appreciated, longitudinal movement of the drive shaft 132 within the outer shaft lumen 108, in turn, slides the housing 148, and thus, the burr 140 along the cutting window 118 between a proximal position (FIG. 15) and a distal position (FIG. 16).

Alternatively, a guidewire 154 may be provided on which the burr 140 can slide, as illustrated in FIGS. 18 and 19. In particular, the guidewire 154 extends along the cutting window 118 and is connected to the distal tip of the sleeve shaft 106 using suitable means, such as welding or soldering. The burr 140 has a lumen (not shown) through which the guidewire 154 extends, and thus, the burr 140 may ride along guidewire 154. The guidewire 154 proximally extends through a lumen (not shown) within the drive shaft 132 and extends out of the handle 120. As can be appreciated, longitudinal movement of the drive shaft 132 within the lumen 108 of the sleeve shaft 106, in turn, slides the burr 140 along the guidewire 154 in the cutting window 118 between a proximal position (FIG. 18) and a distal position (FIG. 19).

Tension is placed on the guidewire 154 in order to prevent the rotating burr 140 from dislodging from the cutting window 118. Notably, in the case wherein the sleeve shaft 106 is composed of a flexible or malleable material, elimination of the rigid burr shaft 142 allows flexing of the distal end 112 of the sleeve shaft 106 along the cutting window 118, if desired. In the illustrated embodiment, the tissue removal element 154 comprises a non-cutting strip 155 formed around the circumference of the burr 140 to prevent the inner surface of the distal end 112 of the sleeve shaft 106 from being damaged by the burr 140 when the distal end 112 is bent upward. The non-cutting strip 155 is preferably composed of a low-friction material, such as Teflon®.

In the embodiments illustrated above, the tissue removal probe 100 comprises a tissue separator 156 formed at the distal end 112 of the sleeve shaft 106. The tissue separator 156 is configured to separate tissue layers distal to the cutting window 118 (e.g., nerves from bone). In particular, the tissue separator 156 comprises an elongated low-profile member 158 that can be precisely located between tissue layers. The tissue separator 156 comprises a blunted spherical tip 160 to prevent cutting of the tissue, thereby facilitating tissue layer separation.

The tissue separator 156 can be manufactured together with the sleeve shaft 106 as one unit. Alternatively, the tissue separator 156 can be manufactured separately. In such case, the tissue separator 156 can be permanently or detachably secured to the distal end 112 of the sleeve shaft 106. For examples, a snap-fit connection, an interference-fit connection, or a screw can be used to detachably secure the tissue separator 156 to the distal end 112.

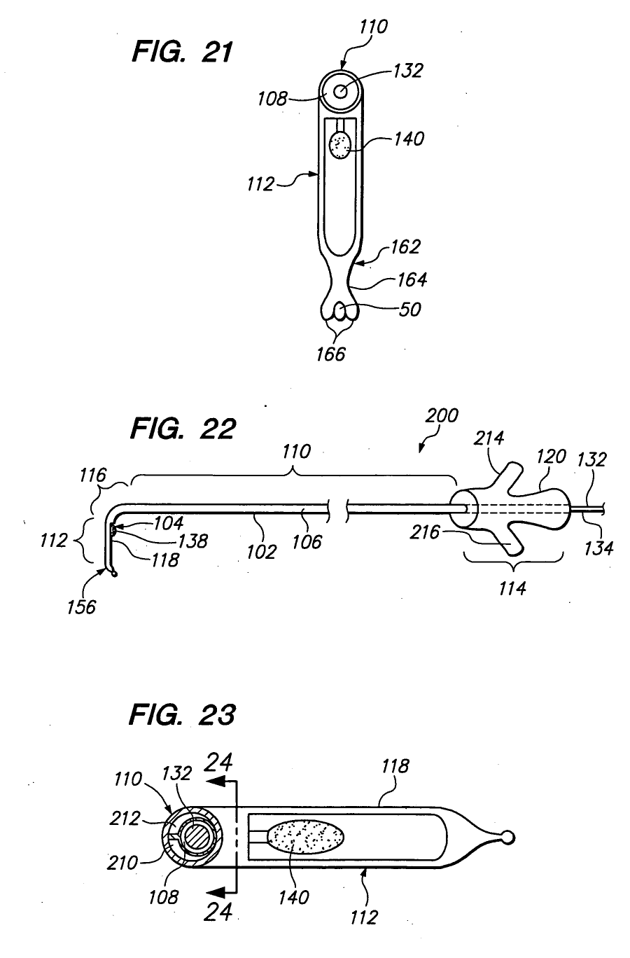

In an alternative embodiment illustrated in FIGS. 20 and 21, the tissue removal probe 100 comprises a tissue separator 162 having a downward curving member 164 and a pair of prongs 166 capable of receiving a nerve or blood vessel 50 therebetween. In this manner, the tissue separator 156 can be guided along the nerve or blood vessel 50 to more easily separate the nerve or blood vessel 50 from other tissue, such as bone. The spacing between the prongs 166 may vary and will depend on a size and shape of the tissue desired to be protected.

The handle 120 is composed of a durable and rigid material, such as medical grade plastic, and is ergonomically molded to allow a physician to more easily manipulate the tissue removal probe 100. The handle 120 has a proximal aperture (not shown) through which the drive shaft 132 extends. The proximal end 114 of the drive shaft 132 can be suitably mated with a drive unit (not shown) configured to both rotate and axially translate the drive shaft 132, and thus, the burr 140 within the cutting window 118. Such drive units are known in the art and will thus not be described in detail here. Notably, if the guidewire 154 is used to guide the burr 140 within the cutting window 118, as shown in FIG. 18, the guidewire 154, which extends through the drive shaft 132, will also be mated to the drive unit. In this case, the drive unit will be configured to continuously hold the guidewire 154 in a pretensed manner.

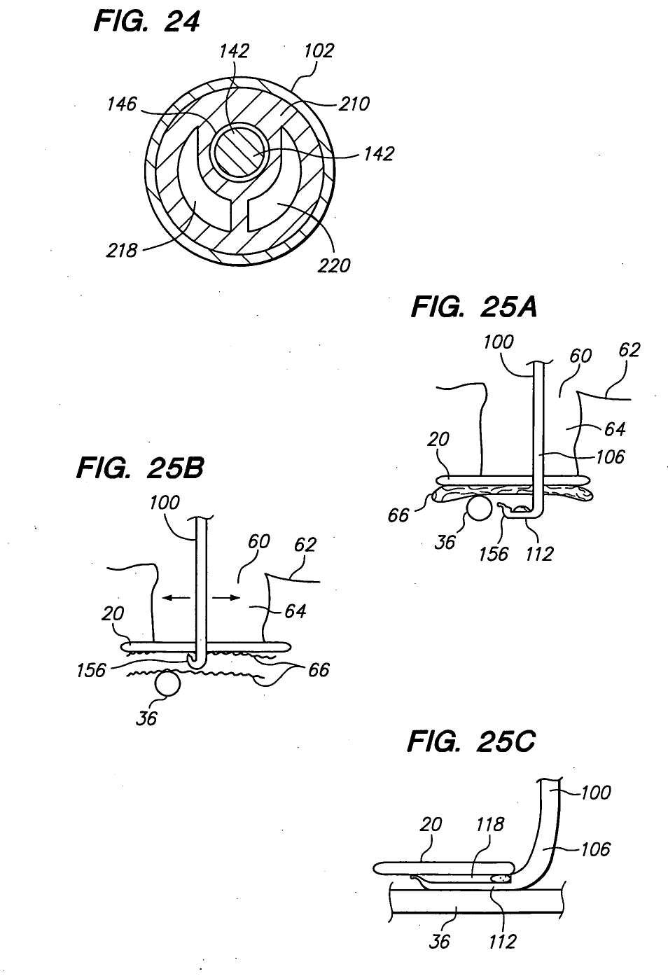

FIGS. 22-24 illustrate another tissue removal probe 200 constructed in accordance with a preferred embodiment of the present inventions. The tissue removal probe 200 is similar to the previously described tissue removal probe 100, with the exception that the tissue removal probe 200 has irrigation and aspiration functionality. In particular, the tissue removal probe 200 comprises respective irrigation and aspiration lumens 210, 212 that extend through the sleeve shaft 106. The irrigation lumen 202 proximally terminates in an irrigation inlet port 206 located on the handle 120 and distally terminates at an irrigation outlet port 218 within the cutting window 118. Likewise, the aspiration lumen 212 proximally terminates in an aspiration outlet port 216 located on the handle 120 and distally terminates at an aspiration inlet port 220 within the cutting window 118. If a bearing 144 is used to slidably support the burr 140, as illustrated in FIG. 24, the respective ports 218, 220 will be formed through the bottom portion of the bearing 144 below the aperture 146. If a guidewire 154 is used instead to slidably support the burr 140, as previously illustrated in FIG. 18, the lumens 210, 212 will extend directly into the cutting window 118 to form the respective ports 218, 220.

Thus, it can be appreciated that the burr 140 can be cooled and/or tissue, e.g., bone particles, can be cleared away from the target site by conveying an irrigation medium, such as, e.g., saline, from an irrigation source (not shown) into the irrigation inlet port 214 located on the handle 120, through the irrigation lumen 210, and out of the irrigation outlet 218 into the cutting window 118. Removed tissue and irrigation fluid can be aspirated from the target site by applying a vacuum to the handle 120 with a vacuum source (not shown), which draws the tissue and irrigation fluid from the cutting window 118, into the aspiration inlet port 220, through the aspiration lumen 212, and out of the aspiration outlet port 216 located on the handle 120.

In the illustrated embodiment, the lumens 210, 212 each has a cross-sectional crescent shape. Alternatively, the lumens 210, 212 can have other cross-sectional shapes, such as circular, elliptical, or other customized shapes. In the illustrated embodiment, the irrigation outlet port 218 and aspiration inlet port 220 are arranged on one side of the sleeve shaft 106 opposite the cutting window 118. It should be noted, however, that the irrigation outlet port 218 and aspiration inlet port 220 can be arranged in other manners. For example, in other embodiments, the irrigation outlet port 218 can be located on one side of the sleeve shaft 106 ninety degrees counterclockwise from the cutting window 118, while the aspiration inlet port 220 can be located on the other side of the sleeve shaft 106 ninety degrees clockwise the cutting window 118.

The importance is that the irrigation outlet port 218 and aspiration inlet port 220 are located near the circumference of the sleeve shaft 106. In this manner, the irrigation fluid is quickly distributed by the rotating burr 140 to the tissue that is to be cut, while the recently cut tissue is aspirated as it comes off of the burr 140. Preferably, if the burr 140 rotates clockwise (as viewed from a distal point), the aspiration inlet port 220 is positioned clockwise relative to the irrigation outlet port 218, such that the tissue is irrigated by fluid exiting the irrigation outlet port 218, then removed by the burr 140, and then aspirated into the aspiration inlet port 220.

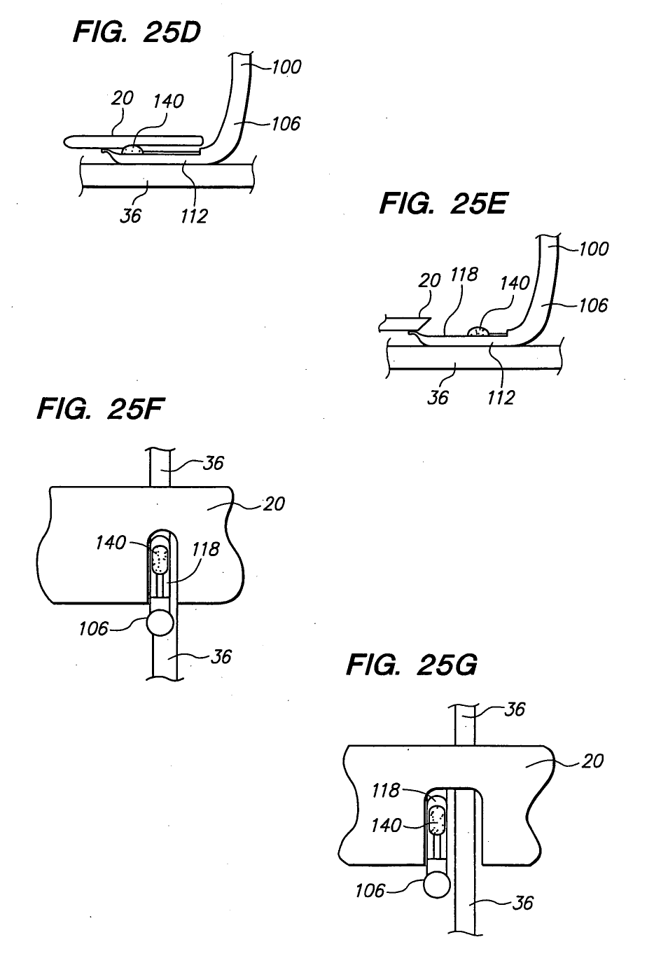

Having described the structure of the tissue removal probe 100, its operation will now be described with reference to FIGS. 25A-25G, in performing a laminectomy. It should be noted, however, that other tissue can also be removed by the tissue removal probe 100. First, the probe 100 is introduced into an incision 60 made in the back 62, and through a passage 64 until the distal end 112 of the sleeve shaft 106 is adjacent the lamina 20 (FIG. 25A). As illustrated, the passage 64 is perpendicular to the plane of the lamina 20, and thus, the ninety degree bent probe 100 is suitable in this case. Alternatively, if flexible or malleable, the distal end 112 of the sleeve shaft 106 can be bent to accommodate the angle between the plane of the lamina 20 and the passage 64. The size of the incision 60 and passage 64 will depend on selected invasiveness of the procedure, but in the illustrated method, an open surgical procedure is used to gain access to the lamina 20. Alternatively, less invasive procedures, such as microsurgical and percutaneous procedures, can be used.

Next, the tissue separator 156 on the distal end 112 of the sleeve shaft 106 is inserted within connective tissue 66 between the nerve root 36 and the lamina 20 and laterally moved in order to separate the nerve root 36 from the lamina 20 (FIG. 25B). In the case where the probe 100 has the pronged tissue separator 162 illustrated in FIGS. 20 and 21, the nerve 36 can be placed in between the prongs 166, such that the tissue separator 162 can be guided inbetween the lamina 20 and nerve 36.

Next, the distal end 112 of the sleeve shaft 106 is placed between the lamina 20 and the nerve root 36, such that the cutting window 118 is placed against the inside surface of the lamina 20 (FIG. 25C). As such, the lamina 20 will be exposed to the tissue removal element 138, and the nerve root 36 will be shielded from the tissue removal element 138.

Next, the drive unit is mated to the handle 120 of the tissue removal probe 102, and operated to rotate and longitudinally translate the burr 140 relative to the window 118. In this manner, a lengthwise portion of the bone tissue along the window 118 is removed without having to move the sleeve shaft 106 (FIG. 25D). The rotating burr 140 can be reciprocated back and forth to complete remove the thickness of the lamina 20 (FIGS. 25E and 25F). The probe 100 can then be laterally moved (or optionally, if having steering functionality, the distal end 112 of the sleeve shaft 106 can be bent to a different location), such that the cutting window 118 is placed against another portion of the lamina 20, and the drive unit operated to remove another lengthwise portion of the bone tissue along the window 118 (FIG. 25G).

Optionally, if the tissue removal probe 200 is used, the removed tissue can be irrigated and aspirated. In particular, an irrigation source and vacuum source can be respectively connected to the irrigation inlet port 214 and aspiration outlet port 216 on the handle 120 of the probe 200. While the tissue removal element 138 is rotated, fluid is conveyed from the irrigation source into the irrigation inlet port 214, through the irrigation lumen 210, and out of the irrigation outlet port 218, where it irrigates the tissue removal element 138. The rotating tissue removal element 138, while being cooled by the irrigation fluid, distributes the irrigation fluid to the tissue within the window 118 while it is being removed, thereby allowing the remove tissue to be more easily aspirated. The rotating tissue removal element 138 forces the irrigation fluid and removed tissue towards the aspiration inlet port 220 where it is aspirated through the aspiration lumen 212, and out of the aspiration outlet port 220 into the vacuum source.

Although particular embodiments of the present inventions have been shown and described, it will be understood that it is not intended to limit the present inventions to the preferred embodiments, and it will be obvious to those skilled in the art that various changes and modifications may be made without departing from the spirit and scope of the present inventions. The specification and drawings are, accordingly, to be regarded in an illustrative rather than restrictive sense. The present inventions are intended to cover alternatives, modifications, and equivalents, which may be included within the spirit and scope of the present inventions as defined by the claims.

1. A tissue removal probe, comprising:
an elongated member having a lumen and a distal end;
a window laterally formed on the member distal end;
a drive shaft rotatably disposed within the member lumen; and
a rotatable tissue removal element disposed on the drive shaft, the tissue removal element being longitudinally slidable within the window.

2. The probe of claim 1, wherein the member is rigid.

3. The probe of claim 1, wherein the member is flexible.

4. The probe of claim 1, wherein the member distal end is curved.

5. The probe of claim 1, wherein the member distal end is steerable.

6. The probe of claim 1, wherein the member distal end is rotatable relative to the remainder of the member.

7. The probe of claim 1, further comprising a bearing affixed within the member lumen, wherein the drive shaft has a rigid distal end slidably disposed within the bearing.

8. The probe of claim 1, further comprising a housing slidably mounted within the window, wherein the tissue removal element is rotatably disposed within the housing.

9. The probe of claim 1, further comprising a guide wire extending along the window, wherein the tissue removal element is configured to slide along the guide wire.

10. The probe of claim 1, wherein the tissue removal element comprises an abrasive burr.

11. The probe of claim 1, further comprising a handle mounted to the member, the handle configured for mating with a drive unit.

12. The probe of claim 1, further comprising a tissue separator disposed on the member distal end.

13. A method of removing tissue, comprising:
providing a probe with a window and a tissue removal element disposed in the window:
placing the window against tissue;
rotating and longitudinally sliding the tissue removal element relative to the window to remove the tissue along the window.

14. The method of claim 13, wherein the tissue is bone tissue.

15. The method of claim 13, wherein the tissue is vertebral lamina bone tissue.

16. The method of claim 13, further comprising placing the window opposite sensitive tissue, wherein the sensitive tissue is shielded from the tissue removal element.

17. The method of claim 16, wherein the sensitive tissue is nerve tissue.

18. A method of removing target tissue without removing non-target tissue, comprising:
providing a probe with a laterally formed window and a tissue removal element disposed in the window;
placing the probe between the target tissue and the non-target tissue, wherein the window is placed against the target tissue and the no-target tissue is shielded from the tissue removal element;
rotating and longitudinally sliding the tissue removal element relative to the window to remove the target tissue along the window.

19. The method of claim 18, further comprising separating the target tissue from the non-target tissue prior to placing the probe therebetween.

20. The method of claim 18, wherein the target tissue is bone tissue.

21. The method of claim 18, wherein the tissue is vertebral lamina bone tissue.

22. The method of claim 21, wherein the non-target tissue is a spinal nerve root.

23. The method of claim 18, wherein the non-target tissue is nerve tissue.
